(19) United States
(12) Patent Application Publication
(10) Pub. No.: US 2025/0137982 A1
(43) Pub. Date: May 1, 2025

(54) Telemetric Sensor System for Sub PPM Hydrogen Detection and Quantification

(71) Applicants: UNM RAINFOREST INNOVATIONS, Albuquerque, NM (US); SensorComm Technologies, Inc., Albuquerque, NM (US)

(72) Inventors: Kannan RAMAIYAN, Albuquerque, NM (US); Kamil AGI, San Francisco, CA (US); C. Jeffrey BRINKER, Albuquerque, NM (US); Lok-kun TSUI, Albuquerque, NM (US); Fernando GARZON, Santa Fe, NM (US); James Albert SMITH, Albuquerque, NM (US)

(21) Appl. No.: 18/925,765

(22) Filed: Oct. 24, 2024

Related U.S. Application Data
(60) Provisional application No. 63/593,044, filed on Oct. 25, 2023.

(51) Int. Cl. G01N 33/00; G01N 27/404; G01N 27/407

(52) U.S. Cl. CPC G01N 33/0014; G01N 27/4045; G01N 27/4074; G01N 27/4075; G01N 33/0036

(57) ABSTRACT

An electrochemical sensor is disclosed, including an electrode disposed on a first ceramic insulation layer, a porous electrolyte stabilization layer disposed around and on top of the electrode, a conductive ceramic substrate in contact with the first ceramic insulation layer on a first side, a second ceramic insulation layer in contact with the conductive ceramic substrate on a second side, and an integrated heater in contact with the second ceramic insulation layer. A telemetric electrochemical sensor system and a method of sensing gaseous analytes is disclosed, which includes exposing a gaseous mixture sample to a hydrogen separation membrane disposed on top of a porous electrolyte which may include an iron (Fe) doped barium niobate perovskite, establishing a first mixed potential at a first electrode, establishing a second mixed potential at a second electrode, and determining a voltage difference as a sensing parameter to be measured.

### CROSS-REFERENCE TO RELATED APPLICATIONS

This application claims the benefit of U.S. Provisional Patent Application No. 63/593,044, filed on Oct. 25, 2023, which is hereby incorporated by reference in its entirety.

### STATEMENT OF GOVERNMENT INTEREST

This invention was made with government support under EEC-1647722 awarded by the National Science Foundation. The government has certain rights in the invention.

### TECHNICAL FIELD

The present teachings relate generally to telemetric sensor systems and, more particularly, to sensitive telemetric sensor systems used in hydrogen detection and quantification.

### BACKGROUND

Hydrogen is a key component of the worldwide effort to reduce greenhouse gas (GHG) emissions and decarbonize industries, however the safe and efficient utilization of hydrogen (H₂) necessitates robust and innovative H₂ monitoring technologies. The current state of the art analytical tools used in the laboratory for H₂ emissions studies include techniques such as mass spectroscopy and optical methods. These methods are expensive, require trained users, and require constant maintenance to keep their optical cavities clean from environmental contamination and may suffer from poor selectivity. Catalytic, thermoelectric, and metal oxide semiconductor sensors are commercially available but suffer from low sensitivity, selectivity, and response drift over time. These properties prevent them from being deployed in continuous-monitoring mode and in outdoor environments where gas infrastructure exists.

Hence there is a need for reliable, robust, cheap, and accurate H₂ sensing technologies to ensure safe handling and utilization in various applications. Implementation of robust H₂ sensors has the potential to stimulate economic growth by creating jobs in future industries reliant on hydrogen. H₂ emissions may also have indirect effects on climate change as emitted H₂ reacts with atmospheric hydroxyl radicals converting them to water vapor, resulting in reduced GHG scavenging as these hydroxyl groups are essential for removing methane and other greenhouse gases, thereby increasing GHG residency time and increasing global warming.

### SUMMARY

The following presents a simplified summary in order to provide a basic understanding of some aspects of one or more embodiments of the present teachings. This summary is not an extensive overview, nor is it intended to identify key or critical elements of the present teachings, nor to delineate the scope of the disclosure. Rather, its primary purpose is merely to present one or more concepts in simplified form as a prelude to the detailed description presented later.

An electrochemical sensor is disclosed. The electrochemical sensor includes an electrode disposed on a first ceramic insulation layer, a porous electrolyte stabilization layer disposed around and on top of the electrode, a conductive ceramic substrate in contact with the first ceramic insulation layer on a first side, a second ceramic insulation layer in contact with the conductive ceramic substrate on a second side, and an integrated heater in contact with the second ceramic insulation layer. Other embodiments of this aspect include corresponding computer systems, apparatus, and computer programs recorded on one or more computer storage devices, each configured to perform the actions of the methods.

Implementations of the electrochemical sensor can include where the electrode may include LSCO, BMNF, BCNF, gold/palladium, platinum, or a combination thereof. The electrochemical sensor may include a first electrode, a second electrode, and a third electrode disposed on the first ceramic insulation layer. The first electrode, the second electrode, and the third electrode operate simultaneously. The first electrode may include LSCO or indium oxide, the second electrode may include a gold-palladium alloy, and the third electrode may include platinum. The electrochemical sensor further may include a gas separation membrane deposited onto the porous electrolyte stabilization layer. The gas separation membrane may include a material configured to transport hydrogen selectively to the first electrode and exclude cross interfering gases. The electrochemical sensor is configured to detect hydrogen, methane, or ammonia in a parts per million range. The electrochemical sensor is configured to detect hydrocarbons, NOx, or CO in a parts per million range. The conductive ceramic substrate may include yttria-stabilized zirconia. The porous electrolyte stabilization layer may include yttria-stabilized zirconia. The electrode disposed on the first ceramic insulation layer may include iron (Fe) doped barium niobate perovskite. The iron doped barium niobate perovskite may include BaMgₙNb_{0.67-x}FeₓO_{3-δ} (BMNF) with a concentration of Fe from about x=0 to about x=0.50 and a concentration of Mg from about n=0.0 to about n=0.50. The iron doped barium niobate perovskite comprises BaCaₙNb_{0.67-x}FeₓO_{3-δ} (BCNF) with a concentration of Fe from about x=0 to about x=0.50 and a concentration of Ca from about n=0.0 to about n=0.50.

A telemetric electrochemical sensor system is disclosed. The telemetric electrochemical sensor system includes a first electrode which may include LSCO, BMNF, or BCNF, a second electrode may include a gold-palladium alloy, and a third electrode may include platinum disposed on a first ceramic insulation layer, a porous electrolyte stabilization layer disposed around and on top of the electrode, a conductive ceramic substrate in contact with the first ceramic insulation layer on a first side, a second ceramic insulation layer in contact with the conductive ceramic substrate on a second side, and an integrated heater in contact with the second ceramic insulation layer. The system also includes where the electrochemical sensor is configured to detect hydrogen, methane, or ammonia in a parts per million range. Other examples of this aspect include corresponding computer systems, apparatus, and computer programs recorded on one or more computer storage devices, each configured to perform the actions of the methods.

A method of sensing gaseous analytes is disclosed, which includes collecting a gaseous mixture sample containing a target analyte, contacting the gaseous sample with a mixed potential electrochemical (MPE) sensor, generating a signal based on a quantity of the target analyte in the gaseous mixture sample to quantify a concentration of the target analyte in the gaseous mixture sample. The method also includes transmitting the concentration of the target analyte to a networked computing device. Other examples of this aspect include corresponding computer systems, apparatus, and computer programs recorded on one or more computer storage devices, each configured to perform the actions of the methods.

Implementations of the method of sensing gaseous analytes can include where the target analyte is hydrogen, methane, alkanes or ammonia, or a combination thereof. A range of the concentration of the target analyte is in a parts per million (ppm) range. Contacting the mixed potential electrochemical (MPE) sensor may include exposing the gaseous mixture sample to a hydrogen separation membrane disposed on top of a porous electrolyte which can include an iron (Fe) doped barium niobate perovskite, establishing a first mixed potential (EMix 1) at a first electrode which may include LSCO, BMNF, or BCNF, establishing a second mixed potential (EMix 2) at a second electrode which may include a gold-palladium alloy, and determining a voltage difference (JE) as a sensing parameter to be measured. Implementations of the described techniques may include hardware, a method or process, or computer software on a computer-accessible medium.

The features, functions, and advantages that have been discussed can be achieved independently in various implementations or can be combined in yet other implementations further details of which can be seen with reference to the following description.

### BRIEF DESCRIPTION OF THE DRAWINGS

The accompanying drawings, which are incorporated in and constitute a part of this specification, illustrate embodiments of the present teachings and together with the description, serve to explain the principles of the disclosure. In the figures:

FIGS. 1A and 1B are schematics of a mixed potential electrode sensor, in accordance with the present disclosure.

FIGS. 2A and 2B are plots showing the response of a 4-element MPE sensor to hydrogen and a methane hydrogen blend, respectively, in accordance with the present disclosure.

FIGS. 3A and 3B are plots of sensor response of a gold electrode with a silicon dioxide hydrogen separation membrane coating and without the silicon dioxide coating, respectively, in accordance with the present disclosure.

FIGS. 4A and 4B are a schematic showing a simple 3-layer artificial neural network for prediction of gas concentration mixtures based on 3 sensor inputs and a plot of concentration error showing <2.5 ppm % error for quantification of CH₄ from simulated natural gas mixtures, respectively, in accordance with the present disclosure.

FIGS. 5A, 5B, and 5C show an integrated wireless sensing package system in a diagram, a vehicle mounted continuous NOx monitoring system positioned in the tailpipe of a car, and mounted on a car, respectively, in accordance with the present disclosure.

It should be noted that some details of the figures have been simplified and are drawn to facilitate understanding of the present teachings rather than to maintain strict structural accuracy, detail, and scale.

### DETAILED DESCRIPTION

Reference will now be made in detail to exemplary embodiments of the present teachings, examples of which are illustrated in the accompanying drawings. Wherever possible, the same reference numbers will be used throughout the drawings to refer to the same, similar, or like parts.

The present disclosure provides an advanced solid-state hydrogen sensor offering unmatched selectivity, accuracy, scalability, and cost-effectiveness. It enhances safety and efficiency across diverse industries, enabling the widespread adoption of hydrogen as a clean energy source. Modest power and supporting electronic needs, low cost, long-lifetime and stable response characteristics all contribute to the value proposition and effectiveness of this hydrogen sensor.

A mixed potential electrochemical (MPE) sensor platform as described herein provides Hydrogen (H₂) leak monitoring at low concentrations, in parts per million (PPM) and in sub-PPM, along with ability to monitor climate modifying H₂ precursor gases such as methane (CH₄) and ammonia (NH₃), various alkanes, or combinations thereof. Additional improvement can be realized to achieve very high selectivity towards H₂ and lower detection limits. The improvements utilize silicon dioxide (SiO₂) gas separation membranes as an overlayer to the porous electrolyte in the MPE sensor to achieve excellent selectivity and sensitivity towards hydrogen. This selectivity reduces the likelihood of false readings and enhances safety and reliability in hydrogen-related applications.

An AI-powered IoT-based mobile NOx monitoring package called “Wi-NOx™” has been developed by SensorComm Technologies (SCT) that is currently being mounted on the tailpipes of automobiles to monitor automotive emissions and to provide operation efficiencies, while monitoring pollution. An interface to an MPE sensor can be developed including an analog signal recording device, power for the sensor and integrated heater, and mobile computing hardware to record the results of sensor measurements.

The development of low part per million (PPM) to parts per billion (PPB) level hydrogen sensing technology with very low cross-sensitivity to inference gases and durability is provided herein. Robust mixed potential electrochemical (MPE) sensors can be utilized for rapid PPM level detection, discrimination and quantification, with alternate approaches to achieve the needed PPB level sensitivity and accuracy. Combining two key innovations with existing microsensor arrays to enhance the sensitivity to H₂ to the required PPB levels for ambient monitoring along with achieving high selectivity to avoid any false alarms is described herein. First is the incorporation of hydrogen separation membranes using self-assembled SiO₂ nanoparticles. The second feature is the development of a family of iron doped barium niobate perovskites that show good catalytic activity towards methane and durability under highly reducing conditions. The incorporation of the hydrogen separation membrane to existing MPE sensors to enhance their hydrogen selectivity while optimizing the electrodes with the barium niobate perovskites to enhance sensitivity is provided.

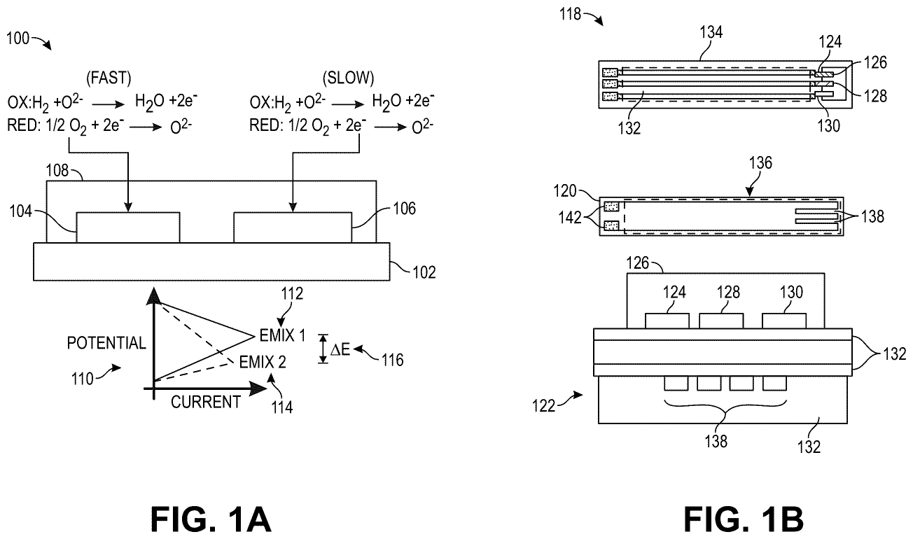

FIGS. 1A and 1B are schematics of a mixed potential electrode sensor, in accordance with the present disclosure.

An MPE sensor 100 as shown, includes two dissimilar electrodes, a first electrode 104 and a second electrode 106 disposed on a substrate 102. Exemplary oxidation and reduction reaction schemes are noted above each of the first electrode 104 and second electrode 106. The first electrode 104 and second electrode 106 are embedded in a solid electrolyte 108 and the operational principle is also illustrated in a plot 110 establishing functioning principle of the MPE sensor 100. On each electrode 104, 106 at steady state, a mixed potential is generated, defined as the potential that is reached when the rates of electrochemical oxidation and reduction reactions are equal in magnitude and no net current is passed between the electrodes. The difference in catalytic activity between the two electrodes causes each electrode to establish a different mixed potential (EMix 1 112 and EMix 2 114), and this voltage difference (ΔE 116) is the sensing parameter which can be readily measured. The present teachings further provide an MPE sensor by applying the hydrogen separation membrane on top of the porous electrolyte to achieve absolute selectivity towards hydrogen. The selective membrane-sensor approach is widely used in ambient temperature gas sensors however, to date, has not been successfully implemented in elevated temperature gas sensors. Similarly, the iron doped barium niobate family has not been explored for hydrogen or methane sensing applications and previous results indicate their activity towards both methane and hydrogen thus demonstrating uses for further improving the sensitivity of MPE sensors.

The MPE gas sensing technology can meet the operational requirements of PPM hydrogen gas emissions monitoring. Further, MPE sensors described herein provide added benefits in terms of simultaneous measurement of the presence of other gases related to hydrogen processing technologies such as CH₄ and CO from steam methane reforming or ammonia from cracking operations. For example, the composition of the electrodes can be fine-tuned for selectivity to the oxidizable or reducible gas species of interest. Au electrodes paired with Pt are known to be selective towards CO, while LaₓSr_{y}CrO₃ electrodes paired with Pt are known to be sensitive to heavier hydrocarbons (C₃H₈) and NOₓ. Indium oxide electrodes using zirconia electrolytes were found to successfully sense H₂ at the ppm level. The UNM team demonstrated the ability of MPE sensors to discriminate between hydrogen and methane for hythane pipeline leak detection using multi-electrode sensors. It is shown that a three electrode MPE sensor consisting of porous yttria-stabilized zirconia (YSZ) electrolytes, electrodes made of La_{0.8}Sr_{0.2}CrO₃ (LSCO), Au—Pd alloys, and Pt, as shown in FIG. 1B, can sense at the 10 ppm level for the hydrocarbon, NOₓ, and CO species that are relevant to automotive emissions analysis. Hence, the integration of these MPE sensors with H₂ membrane micro-separation technology will allow for the development of sensor elements with unprecedented selectivity coupled with greater sensitivity to H₂ and carry out further testing at a wider range of concentrations for hydrogen and hythane mixtures.

The schematics of FIG. 1B include a top view 118 of the MPE sensor 100, a bottom view 120 of the MPE sensor 100, and a side view 122 of the MPE sensor 100. As shown in FIG. 1B, the locations and placements of the various components of the MPE sensor 100 are shown, including a lanthanum strontium cobalt oxide (LSCO) electrode 124, a gold-palladium (AuPd) electrode 128, and a platinum (Pt) electrode 130, surrounded by a porous yttria-stabilized zirconia (YSZ) layer 126. The electrodes 124, 128, 130 are disposed on one of several ceramic insulator 132 layers surrounding the substrate 140 towards both the front sensor side 134 and on the rear heater side 136. Also on the heater side are platinum (Pt) heater elements 138 and several additional electrodes 142 in contact with the heater elements 138. In examples, the electrolyte can be comprised of a porous material. Examples of suitable electrolyte materials can include yttria stabilized zirconia (YSZ) with varying yttrium concentrations such as 3YSZ and 8 YSZ and Lanthanum Strontium Gallium Magnesium Oxide (La_{0.80}Sr_{0.20}Ga_{0.80}Mg_{0.20}O₃₋ₓ). Alternate examples of electrode materials can include metals or metal alloys, including, but not limited to Fe doped barium magnesium niobates, Fe doped barium calcium niobates where Fe concentration vary from 0 to 0.7 and Ca or Mg concentrations vary from 0 to 0.50. The barium calcium niobates are double perovskites with space group of FM3M while the barium magnesium niobates belong to PM3M space group. Additional substrates for MPE sensors of the present disclosure can include but are not limited to Mg stabilized zirconates (MSZ), MgAlO₄ spinel and combinations of MSZ or YSZ layers with top coating of MgAlO₄. In examples, one or more of the electrodes can include LSCO, BMNF, BCNF, gold/palladium, platinum, or a combination thereof.

The current state of the art analytical tools used in the laboratory for H₂ emissions studies include sample gas drying and mass spectroscopy or catalytic oxidation of hydrogen to water and subsequent detection in cavity-ring down and mid IR spectrometers which have concentration sensitivity at the ppb level. These instruments are expensive (˜$30K-100K), require trained users, and require constant maintenance to keep their optical cavities clean from environmental contamination. These properties prevent them from being deployed in the continuous-monitoring, outdoor environments where gas infrastructure exists. Catalytic sensors are capable of high concentration detection but lack the ability to detect at low concentrations. Metal oxide sensors based on the conductivity changes in the presence of an oxidizable gas have been demonstrated and can produce sensitivity of detection at the 10-100 ppm level, but they experience signal drift over time and require recalibration that has prevented their use for long-term monitoring. Pellistor-type hydrogen sensors use the catalytic combustion of hydrogen to create a temperature difference between an active Pt bead and a poisoned reference Pt bead that is measured as the signal. Despite being a well-developed commercial technology, pellistor-type sensors lack selectivity between combustible gases such as hydrocarbons and CO. A comparison of key characteristics of these sensors with the presently disclosed MPE sensor technology is given in Table 1. The lack of sensor technologies for widescale deployment to monitor the hydrogen leak necessitates the further development of MPE sensor technology to produce reliable data from remote locations inaccessible to regular human monitoring. The MPE sensors of the present teachings coupled with machine learning technique has achieved high sensitivity and selectivity. The integration of the present MPE sensors with H₂ membrane micro-separation technology provides a unique advantage and can make these sensors a market leader in hydrogen sensing for industries associated with hydrogen production, transport, and utilization.

TABLE 1
A number of different sensing technologies are used to detect hydrogen
leaks and these are compared with the disclosed mixed potential sensors.

| | | Lab | H₂ | Catalytic | SC | Mixed |
|---|---|---|---|---|---|---|
| | | (GC/MS/ | oxidation + | Gas | Gas | Potential |
| Technology | Airborne | CRDS) | Portable IR | Sensors | Sensors | Sensors |
| Cost | Very | High | Med | Low | Low | Low |
| | High | ($100 k+) | ($30 k) | | | |
| Area (sq) | Miles | Feet | Feet | Feet | Feet | Feet |
| Resolution | | | | | | |
| Sensitivity | ppb | ppb | ppb | ~1% LEL | ppm | ppm |
| Robustness | N/A | Low | Low | Med | Med | High |
| Selectivity | Yes | Yes | Yes | No | No | Yes |
| Stability | N/A | frequent | frequent | Sensor | Sensor | High over |
| | | maintenance | maintenance | drift | drift | 1000 hrs |
| Size, | Very | Large, 3- | Medium, | Small, | Small, | Small, |
| weight, | Large | 20 kg, | kg, | g, W | g, W | g, W |
| power | | 10-100 s W | 10 s W | | | |

The second key element of the present disclosure includes two sets of iron doped barium niobate perovskites, BaMg_{0.33}Nb_{0.67-x}FeₓO_{3-δ} (BMNF) and BaCa_{0.33}Nb_{0.67-x}FeₓO_{3-δ} (BCNF) where Fe concentration is varied from x=0.0 to x=0.50 and magnesium or calcium concentration is from about n=0 to about n=0.50, and in one example, BaMg_{0.33}Nb_{0.67-x}FeₓO_{3-δ} or BaCa_{0.33}Nb_{0.67-x}FeₓO_{3-δ}. Both these materials show excellent catalytic activity towards methane activation and hydrogen production at temperatures relevant for the present MPE sensor operation. Both BMNF and BCNF showed excellent thermochemical stability under highly reducing and coke forming conditions. Fe doping induces their catalytic activity which can be used as a leverage to fine-tune their catalytic activity while Nb changes its oxidation state between Nb⁴⁺ and Nb⁵⁺ that is observed to be the key for their chemical stability. These materials show high promise as an electrode on MPE sensors for detecting methane or hydrogen while providing an opportunity for a new class of highly sensitive electrodes.

Prior work enabled the development of multi-gas sensors and the PPM level MPE sensors developed for H₂, or for interference gases. Sub-PPM sensitivity is necessary to develop an effective early warning system for leaks. In hythane (hydrogen/methane mixtures in existing pipelines), hydrogen is more likely to leak before methane due to its smaller size (8 times smaller than methane). In addition, the low concentration of hydrogen in hythane further necessitates higher sensitivity towards hydrogen. The proposed disclosure leverages previous work to develop sub-PPM sensitivity systems for hydrogen/methane leak detection and release quantification. Developments in H₂ separation membrane technology can also be leveraged to improve sensitivity and selectivity. The selective membrane-sensor approach has not been successfully implemented in elevated temperature gas sensors hence this provides an opportunity for new developments. For example, the gas separation membrane in MPE sensing applications described herein can include a material configured to transport hydrogen selectively to the working electrode and exclude cross-interfering gases, or gases that are not the analyte of interest. Exemplary materials can include but are not limited to (i) metallic membranes such as palladium (Pd), vanadium (V), tantalum (Ta), niobium (Nb), titanium (Ti), and alloys of these metals that selectively transport hydrogen, (ii) inorganic ceramic microporous membranes made of Vycor® silica glass, alumina, glass, silica, zeolites, and metal organic frameworks (MOFs), (iii) carbon based molecular sieving membranes, and (iv) polymeric membranes such as polysulfone, polyimide and polyamides, or combinations thereof. It should further be noted that the working electrode can refer to any electrode where the investigated processes occur, in this example, the detection and leading to the generation of either of the mixed potentials described previously. Furthermore, while the generation of the mixed potential occurs at the electrodes, the detection of the mixed potentials is done by external hardware such as, but not limited to a voltmeter.

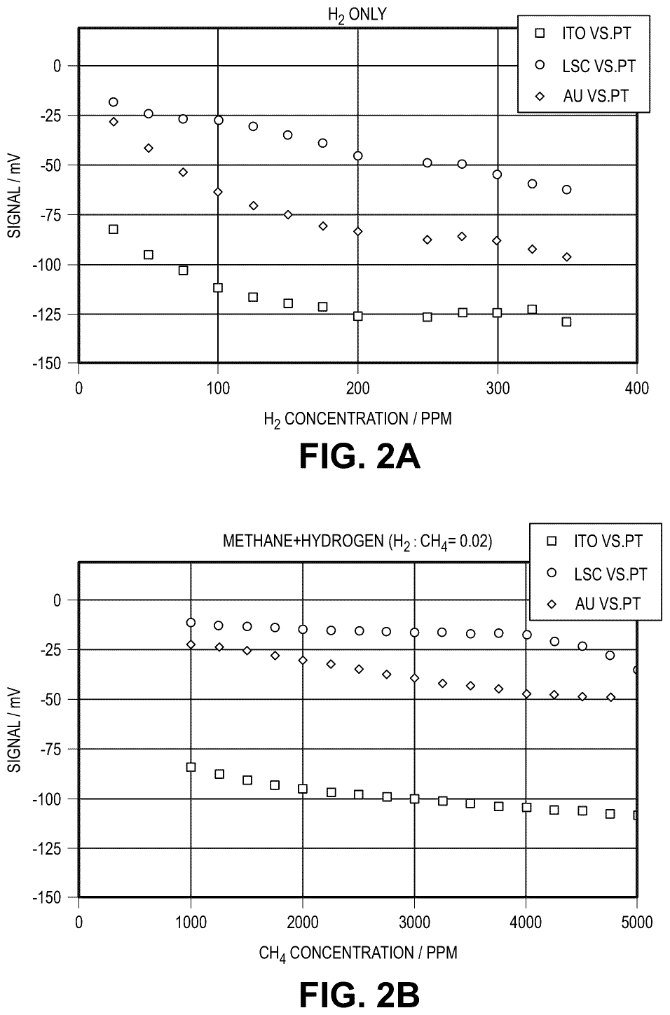

FIGS. 2A and 2B are plots showing the response of a 4-element MPE sensor to hydrogen and a methane hydrogen blend, respectively, in accordance with the present disclosure. Mixed potential electrochemical (MPE) gas sensing technology can be shown to meet the operational requirements of parts per million hydrogen gas emissions monitoring. The ability of MPE sensors to discriminate between hydrogen and methane for hythane pipeline leak detection using multi-electrode sensors has been demonstrated. The sensitivity of the demonstrated device was greater than 35 mV/PPM at low hydrogen partial pressures. Preliminary results show it is suitable for hydrogen identification and quantification even in the presence of common interference gases such as methane. FIG. 2A shows that the limit of detection for H₂ in air is below 20 PPM. MPE sensors were tested with H₂:CH₄ mixes from 1000-5000 PPM at a fixed H₂:CH₄ ratio of 0.02 as shown in FIG. 2B. A previous demonstration utilizing a three electrode MPE sensor consisting of porous yttria-stabilized zirconia (YSZ) electrolytes, electrodes made of La_{0.8}Sr_{0.2}CrO₃ (LSCO), Au—Pd alloys, and Pt (FIG. 1B) was shown to sense at the 10 ppm level for the hydrocarbon, NOₓ, and CO species that are relevant to automotive emissions analysis. This device features an integrated heating element which uses resistive heating to bring the device to the 400-600° C. operating temperature. It has been shown that these sensors exhibit stable responses while being exposed to NOₓ/NH₃ mixtures on the order of 100 days, others have examined the response of MPE sensors in engine dynamometer testing and found that the sensor withstood exposure to exhaust gases as well as demonstrated the ability to resolve changes in engine conditions with resolution on the order of seconds. Based on these results and previous investigations by the authors it is clear that the durability of these sensors exceed over 1000 hours of continuous operation and testing. Hence, it can be concluded that MPE sensors can meet the needs of both long-term stability in harsh conditions with substantially higher endurance than optical spectroscopy systems. Fast response times demonstrated in these results will also lead to an early warning for a leakage event. The manufacturing of these sensors using traditional screen printing technologies in similar packaging to the lambda oxygen sensors that are widely used in vehicles today also shows that there is a reasonable path towards low-cost mass production of these devices for widespread deployment.

Development of micro-separation membranes for hydrogen gas purification, and in particular, the development of inorganic membranes involves a sequential deposition of alumina particles (3-5 μm, 1-2 μm, and 200 nm) in the descending particle size order on a porous alumina that provides structural support. After the deposition of the 200 nm alumina particle layer, another layer of 60 nm TiO₂ is applied to smooth out the surface for the silica top layer. SiO₂ particles are deposited on these layers to form a microporous network with average pore size of about 0.3 nm that is key for hydrogen separation.

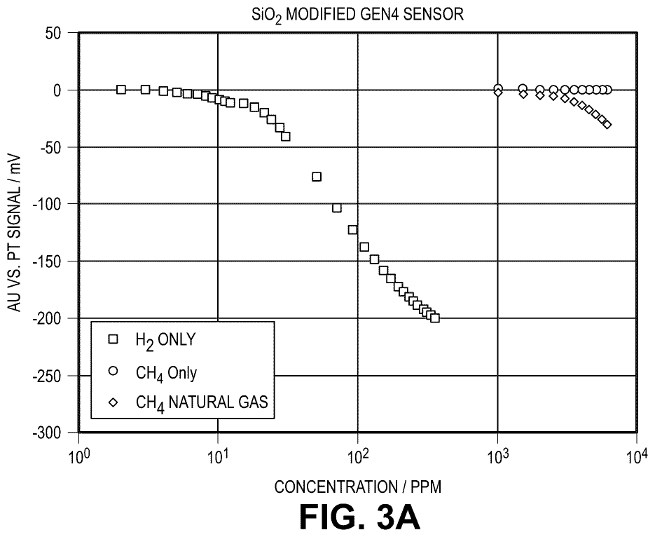

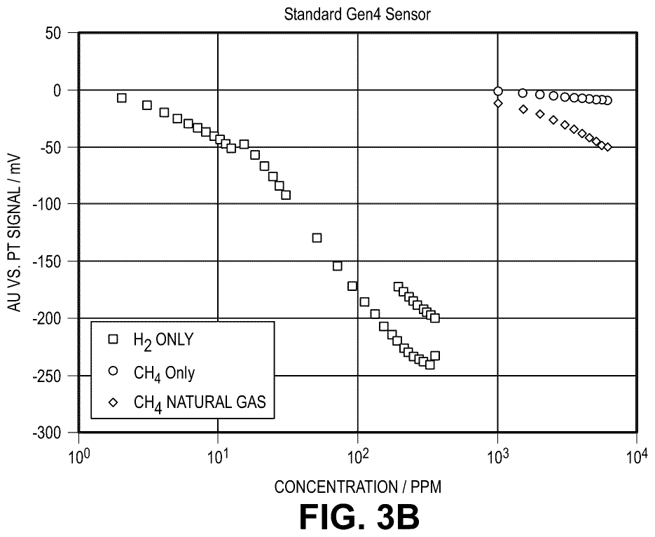

FIGS. 3A and 3B are plots of sensor response of a gold electrode with a silicon dioxide hydrogen separation membrane coating and without the silicon dioxide coating, respectively, in accordance with the present disclosure. A challenge in integrated SiO₂ membranes to the porous YSZ electrolyte is that SiO₂ is known to interact with YSZ electrolytes at very high temperatures and reduce their ionic conductivity at the grain boundary, acting as a poison. To evaluate the feasibility of the overall approach, a thin SiO₂ film was grown using atomic layer deposition (Picosun R-150P) on top of MPE sensor structures and the MPE sensor response to hydrogen, methane and natural gas with (FIG. 3A) and without (FIG. 3B) the SiO₂ coating is shown. Clearly, the MPE sensor retains its sensitivity towards hydrogen after SiO₂ membrane coating while the sensor response to methane and natural gas is decreased comparatively. The overcoated device as compared to the reference device, shows much improved quantitative sensitivity at the <10 PPM hydrogen concentrations. This preliminary result shows the potential for this approach. However, further measurements are required to understand and optimize the process of hydrogen separation membrane coating on the porous YSZ electrolyte. There is a gap in understanding regarding the interaction of SiO₂ nanoparticles with YSZ mainly at the low operating temperatures associated with the sensor operation (400-600° C.). The role of ceramic SiO₂ membrane coating thickness, coating rate, sintering temperature and time, cooling rate are all needs to be optimized. Similarly, further experiments may be required to understand if the TiO₂ layer is needed to be on the porous YSZ surface to smooth the surface before SiO₂ layer is deposited.

Both BMNF and BCNF perovskites have been tested as electrode materials in methane to ethylene conversion where the electrode inks to produce these porous electrodes are brush painted on a dense electrolyte. However, the electrode formulations of the present MPE sensors employ a dense electrode deposited on an inactive dense ceramic substrate followed by deposition of porous YSZ electrolyte. Optimization of the electrode ink formulations, sintering conditions and variation of concentrations of Fe doped BMNF and BCNF perovskites can provide improved electrodes. Additive manufacturing for fabricating these MPE devices can also be utilized in this regard.

Additive manufacturing (AM) of ceramics and metals by extrusion or ink deposition is a technology that enables the rapid prototyping of materials relevant to MPE sensors. The additive manufacturing of LSCO/YSZ/Pt MPE sensors has been previously demonstrated along with their ability to detect hydrocarbons, NOₓ, and NH₃ at the 100 ppm level as well as multi-electrode MPES sensors with sensitivity of 1000 ppm towards CH₄ in natural gas. It is possible to print a range of metals (i.e. Au), metal alloys (Au—Pd), along with BMNF and BCNF ceramic electrode materials and identify which materials are most sensitive for detection of hydrogen in gas processing environments with the goal of sensitivity at the 10-1000 PPB level. screen printing of best performing materials into a multi-electrode MPE sensor can also be done. A suitable set of materials can be optimized by additive manufacturing, followed by a transition to traditional screen-printing methods for scaled up production of sensors.

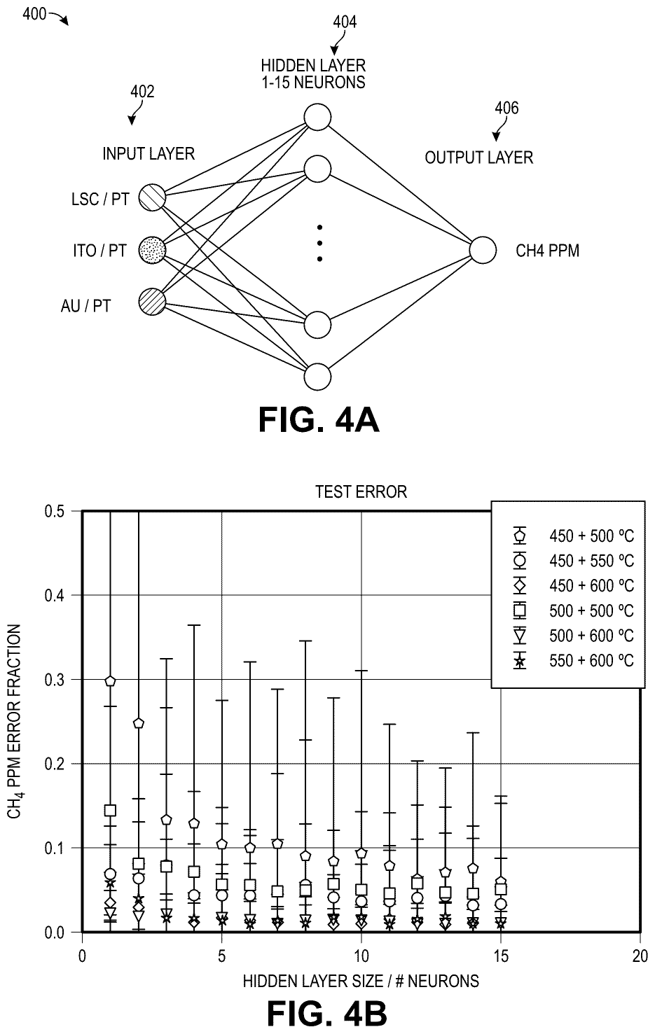

FIGS. 4A and 4B are a schematic showing a simple 3-layer artificial neural network for prediction of gas concentration mixtures based on 3 sensor inputs and a plot of concentration error showing <2.5 ppm % error for quantification of CH₄ from simulated natural gas mixtures, respectively, in accordance with the present disclosure. The quantification of a single gas species in a carrier gas represents one level of complexity for many sensor technologies, but the introduction of more than one combustible gas introduces complications related to sensor cross interference. Quantification and discrimination of various gases has been attempted with the use of so called “artificial noses” that use multiple sensing elements. A machine learning algorithm (MLA) approach to this challenge is of interest because of recent developments in availability of portable computing hardware for deployment in internet of things (IoT) platforms. A number of approaches involving Bayesian techniques have been studied to deconvolve the signals received from multi-element devices including MPE sensors, though these require substantial human input and tuning. A range of MLAs for natural gas detection were recently benchmarked, finding that a random forest algorithm resulted in a computationally efficient yet high accuracy method for discriminating signals associated with natural gas from other interferent sources. Artificial neural networks (ANN) already widely deployed in image analysis, natural language processing, and speech recognition are a powerful MLA capable of automatically learning relations between sensor signals and gas concentrations or mixture labels. The schematic of a 3-layer artificial network 400 in FIG. 4A shows an input layer 402 having multiple electrodes, a hidden layer 404 having from about 1 to about 15 neurons, and an output layer 406. Quantification of binary and ternary mixtures of C₃H₈, NOₓ, CO, and NH₃ using artificial neural networks were shown to have as high as 5-10 ppm level of accuracy in concentrations between 10-250 ppm. Recent efforts have also demonstrated the application of ANNs to natural gas quantification with >97.5% ppm accuracy (as shown in FIG. 4B).

Accuracy and processing time can be studied based on a wide range of MLAs trained to identify mixtures of H₂ with other interferent gases, and quantify concentrations of hydrogen in air and both manmade/naturally occurring interferents. Simulated gas mixtures can be used initially and the results validated on sampling of hydrogen gas and interferents. The performance of the MLAs can be evaluated during a limited field test. These MLAs will initially be trained and operated on commercially available low-cost Next Unit of Computing (NUC) systems but will ultimately be integrated into a combined sensor, computing hardware, and wireless communication package.

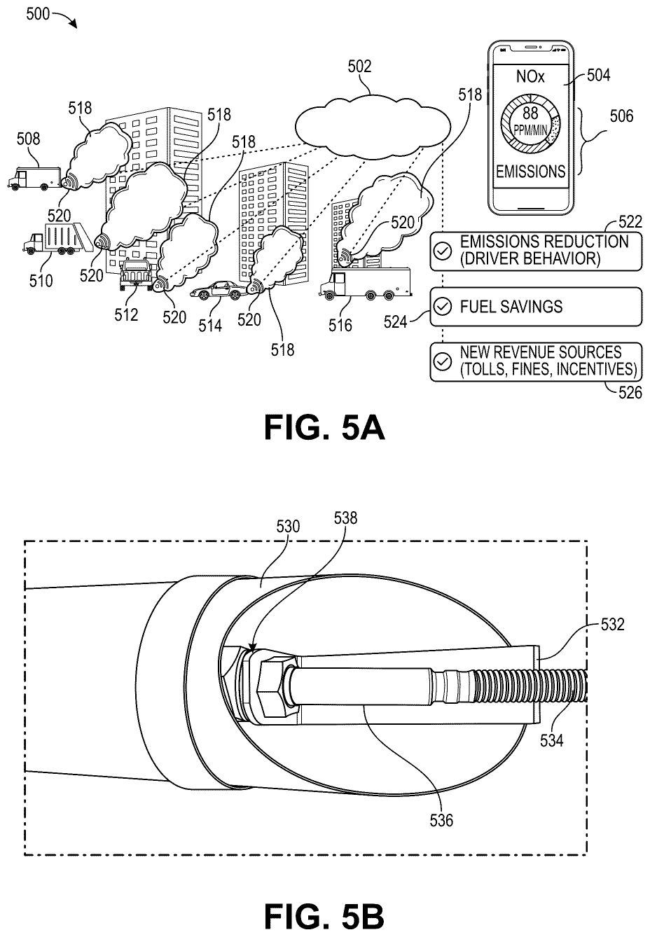

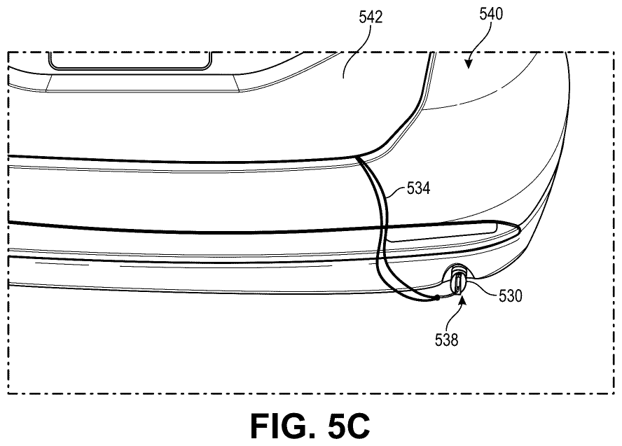

FIGS. 5A, 5B, and 5C show an integrated wireless sensing package system is diagram, a vehicle mounted continuous NOₓ monitoring system positioned in the tailpipe of a car, and mounted on a car, respectively, in accordance with the present disclosure. An Integrated Wireless Sensing Package (SensorComm Technologies) is shown, which is an AI-powered IoT-based mobile NOₓ monitoring package called “Wi-NOₓ™” that can be utilized for monitoring the tailpipes of automobiles to study automotive emissions. This real-time pollution monitoring system 500 includes a schematic showing example hardware and elements. The real-time pollution monitoring system 500 encompasses the gas sensor 538, electronics hardware, and wireless communication over cellular data networks to perform city-wide emissions analysis with the objective of integrating such technologies into smart cities of the future. Shown in FIG. 5A is an arrangement of several types of target vehicles, including a delivery vehicle 508, a waste vehicle 510, a mass-transit vehicle 512, a fleet/consumer vehicle 514, and a transport vehicle 516. Each vehicle includes a gas sensor based on the sensors and systems as described herein, with a capability for wireless transmission 520 to a data cloud 502, in which is managed data surety, spectrum sensing information, machine learning, and artificial intelligence elements. The data transmitted from the one or more vehicles 508, 510, 512, 514, 516 provides information on exhaust or pollution 518 produced by each vehicle. The data can then be analyzed and transmitted or sent to a device which is configured to read out or display data readout and analysis 506 results. In additional steps, the information or data can be utilized to take action on emissions reduction 522 by understanding and influencing driver behavior, fuel savings 524, or generate new revenue sources 526 in the form of tolls, fines or incentives. FIGS. 5B and 5C are photographs showing the location of an exhaust pipe 530, whereby a clamp 532 is fixed to guide a data cable and/or sensor cable 534 to a sensor housing 536, which includes a gas sensor 538. The gas sensor 538 can be mounted on a vehicle 540, in most cases near the rear of a vehicle 542, in proximity to the exhaust pipe 530.

The present disclosure provides the development of a versatile hydrogen sensor technology with integrated data collection and internet telemetry for emission monitoring in the emerging hydrogen economy that require hydrogen emissions monitoring during production, transportation, distribution and utilization for safe operation. The sensor system combined with advanced machine learning data processing, can enable the rapid and continuous identification, quantification, and location monitoring through data transmission of hydrogen leaks and serve as an early warning system for pipeline leakage and to quantify the loss of product and air pollution. This can also include the development of sensing elements specifically suited for detection of hydrogen and subspecies gases present in typical hydrogen gas processing, transportation and distribution environments. Hydrogen separation membranes can be used to improve selectivity and identify hydrogen leaks in various environments. This can provide or produce a complete sensing package that will consist of a multi-electrode MPE sensor, electronics hardware to power and take measurements of the sensors, computing hardware to run pre-trained MLAs to perform identification of whether a signal set is associated with a hydrogen gas leakage event or some other interferent and provide feedback through wireless communication systems to help pinpoint the leak. Thus, a market ready sensor combined with control and measurement electronics, integrated data processing and internet capable communications.

### Materials Optimization of Sensor System for Hydrogen Detection

Sensor prototyping can produce MPE sensors constructed by deposition of electrodes with additive manufacturing or physical vapor deposition on to ceramic substrates and a layer of porous electrolyte will be deposited on top and around one or more of the electrodes. Individual layers of SiO₂ membrane or a combination of TiO₂ and SiO₂ membrane are coated on top of the porous electrolyte by infiltration or by atomic layer deposition (ALD). Hydrogen separation membrane coating thickness, coating rate and cooling rate can be optimized to fabricate the sensor with highest hydrogen sensitivity and selectivity. This fabrication can result in the production of at least one 2-electrode sensor which can detect hydrogen at the >1-1000 PPM level.

The sensor performance evaluation of systems including MPE sensors with and without the SiO₂ membrane coating can be done by exposing the sensors to hydrogen and gas mixtures containing hydrogen and other combustible gases and their response is analyzed. Selectivity and cross interference studies in multi-species mixtures and electrochemical studies can interrogate the kinetics of the gas phase reactions and gas diffusion on these devices. This step can provide at least one 3-electrode sensor with different sensing elements that can detect hydrogen and another interferent gas at the >1-1000 PPM level.

Multi-electrode MPE sensor elements can include at least three sensing element pairs identified for sensitivity and selectivity to H₂, CH₄, CO, NH₃, and CO₂ at the 1-1000 ppm level.

In examples, both BMNF and BCNF perovskite materials can be used for H₂, CH₄, and NH₃ sensing application for PPB to PPM level concentrations. The ink formulation for the niobates can be optimized for the best performing catalyst. The selectivity of this type of material towards individual gases can be evaluated with and without the optimized SiO₂ membrane coating on MPE sensor.

### Machine Learning Algorithms (MLAs) for Sensor Deconvolution

MLAs can be trained on the sensor data obtained during testing. The algorithms will be trained to categorize qualitatively whether a signal detected by the sensor is hydrogen gas or an interferent and report quantitative concentration values. Training data in the form of sensor signals and programmed concentration values determined by a multi-channel gas mixer can be collected by exposing a multi-electrode MPE sensor to varying concentrations of H₂ and interferents such as CH₄, CO, and NH₃. This can use H₂ as a single gas mixture using an air carrier gas at concentration levels of 10 PPB-1000 PPM and then 1 PPB-1000 PPM. Binary and ternary mixtures of H₂ with known secondary species such as CH₄, NH₃. CO and CO₂ can be added in concentrations representative of hydrogen gas or interferents. Mixtures can be chosen to simulate industrially relevant hydrogen blends. The sensitivity to specific gases can be measured under open circuit and with tuning techniques such as constant current biases and following the use of applied potential pulses.

This can further provide a labeled dataset will be collected which can be used to begin training MLAs at the 10 PPB-1000 PPM level. The data can include sensor readings, gas concentrations and categories. The steps include the implementation, training, and optimization of MLAs based on the dataset collected at the end of technology maturation plan to quantify hydrogen concentrations in an air background in a range of 10 PPB-1000 PPM levels. These algorithms include but are not limited to polynomial regression models, artificial neural networks, support vector machines, and forest/ensemble approaches. Sensors utilizing such aspects should achieve >95% concentration accuracy for datasets (1 PPM to 1000 PPM levels). The implementation, training and optimization of machine learning algorithms based on the dataset collected from multi element sensors can be used to quantify hydrogen and interferent gas concentrations in a range of 10 PPB to 1000 ppm. Exemplary systems should achieve >95% concentration accuracy for datasets for H₂ and interferents such as CH₄ and NH₃ (10 PPB to 1000 PPM). The MLAs can further be optimized for inference at runtimes of <0.1 s per point on portable hardware.

### Development of an Integrated Wireless Sensing Platform [SCT]

An integrated hardware package consisting of a multi-electrode MPE sensor, electronics to obtain sensor readings, perform MLA inference using pre-trained networks, and transmit the results using wireless communication is described herein. An interface to an MPE sensor can include an analog signal recording device, power for the sensor and integrated heater, biasing algorithms and mobile computing hardware to record the results. Such a hardware package can include the capability to operate a two-electrode element in a laboratory test setup using portable data acquisition hardware with mV level precision.

Example systems include the capability to operate multi-element sensor designs with at least 3 electrode pairs simultaneously, remotely transmit the data via cellular networks, and operate this device without the need for wired power. Mobile computing hardware suitable for the power and a form factor for a portable sensing device are selected and integrated into the sensing platform. MLAs may need to be further evaluated to be operative on the selected hardware and their results will be compared with the results on desktop PCs, general purpose single board computers, or commercially available mobile machine learning development boards. The completed sensor package includes a portable power supply, sensor reading and operational electronics, MLA inference hardware, and wireless communication over cellular network to remotely relay the results.

### Field Test and Validation

Field test and validation is used to assess conditions in the field that may affect measurements, validate the sensing capability of the combined SiO₂ membrane/MPE sensor/MLA inference system, and test the standalone wireless sensing and communications package. Field testing includes a first portion to gather sensor readings for ambient hydrogen and other subspecies emissions present at the site during nominal day-to-day operations and validation with sampling and laboratory/field gas analysis tools such as mass spectroscopy and mid-IR optical absorption spectrometer. The influence of distance from equipment can be studied to determine the necessary density of sensing elements that will be needed to provide pipeline coverage. Impact of local wind conditions and response times to events such as gas flaring can be of particular importance. Information about long term sensor reading stability in the field test environment can also be informative. The sensor package can be further tested to demonstrate the ability to identify a gas release event at a field test site. The system is exposed to gases sampled at the site, and interferent simulants are brought to the site. This manner of test validates whether the sensor package itself is also robust against changes in environment outside laboratory testing conditions. Subsequently, the ability to remotely receive a sensor reading, ANN inference data of gas type identification, reporting of species concentration, and continuous monitoring for the course of a minimum of one week can be demonstrated.

The successful demonstration of the AI-powered, IoT-based early warning system for hydrogen fills a key gap in the current market and creates an enabling system where hydrogen sensing is possible in the presence of interference gases. This allows new applications in pipeline leak detection for hythane, hydrogen-based vehicular monitoring and even process optimization for “colored” hydrogen generation (green, blue, brown, etc.) technologies, including the hydrogen hub efforts. With a significant push in the migration to hydrogen infrastructure, safety concerns are becoming important as well. Hydrogen is more flammable than other gases so finding the smallest leaks becomes imperative. In addition, there are questions of the impact of hydrogen leaks, in that it may prolong the presence of methane in the atmosphere. Therefore, proactive thresholds for emissions must be established and monitoring early warning systems will be required.

While the present teachings have been illustrated with respect to one or more implementations, alterations and/or modifications may be made to the illustrated examples without departing from the spirit and scope of the appended claims. For example, it may be appreciated that while the process is described as a series of acts or events, the present teachings are not limited by the ordering of such acts or events. Some acts may occur in different orders and/or concurrently with other acts or events apart from those described herein. Also, not all process stages may be required to implement a methodology in accordance with one or more aspects or embodiments of the present teachings. It may be appreciated that structural objects and/or processing stages may be added, or existing structural objects and/or processing stages may be removed or modified. Further, one or more of the acts depicted herein may be carried out in one or more separate acts and/or phases. Furthermore, to the extent that the terms “including,” “includes,” “having,” “has,” “with,” or variants thereof are used in either the detailed description and the claims, such terms are intended to be inclusive in a manner similar to the term “comprising.” The term “at least one of” is used to mean one or more of the listed items may be selected. Further, in the discussion and claims herein, the term “on” used with respect to two materials, one “on” the other, means at least some contact between the materials, while “over” means the materials are in proximity, but possibly with one or more additional intervening materials such that contact is possible but not required. Neither “on” nor “over” implies any directionality as used herein. The term “conformal” describes a coating material in which angles of the underlying material are preserved by the conformal material. The term “about” indicates that the value listed may be somewhat altered, as long as the alteration does not result in nonconformance of the process or structure to the illustrated embodiment. The terms “couple,” “coupled,” “connect,” “connection,” “connected,” “in connection with,” and “connecting” refer to “in direct connection with” or “in connection with via one or more intermediate elements or members.” Finally, the terms “exemplary” or “illustrative” indicate the description is used as an example, rather than implying that it is an ideal. Other embodiments of the present teachings may be apparent to those skilled in the art from consideration of the specification and practice of the disclosure herein. It is intended that the specification and examples be considered as exemplary only, with a true scope and spirit of the present teachings being indicated by the following claims.

What is claimed is:

1. An electrochemical sensor, comprising:
an electrode disposed on a first ceramic insulation layer;
a porous electrolyte stabilization layer disposed around and on top of the electrode;
a conductive ceramic substrate in contact with the first ceramic insulation layer on a first side;
a second ceramic insulation layer in contact with the conductive ceramic substrate on a second side; and
an integrated heater in contact with the second ceramic insulation layer.

2. The electrochemical sensor of claim 1, wherein the electrode comprises LSCO, BMNF, BCNF, gold/palladium, platinum, or a combination thereof.

3. The electrochemical sensor of claim 1, wherein the electrochemical sensor comprises a first electrode, a second electrode, and a third electrode disposed on the first ceramic insulation layer.

4. The electrochemical sensor of claim 3, wherein the first electrode, the second electrode, and the third electrode operate simultaneously.

5. The electrochemical sensor of claim 3, wherein:
the first electrode comprises LSCO or indium oxide;
the second electrode comprises a gold-palladium alloy; and
the third electrode comprises platinum.

6. The electrochemical sensor of claim 3, wherein the electrochemical sensor is configured to detect hydrogen, methane, or ammonia in a parts per million range.

7. The electrochemical sensor of claim 3, wherein the electrochemical sensor is configured to detect hydrocarbons, NOₓ, or CO in a parts per million range.

8. The electrochemical sensor of claim 1, wherein the conductive ceramic substrate comprises yttria-stabilized zirconia.

9. The electrochemical sensor of claim 1, wherein the porous electrolyte stabilization layer comprises yttria-stabilized zirconia.

10. The electrochemical sensor of claim 1, wherein the electrode disposed on the first ceramic insulation layer comprises iron (Fe) doped barium niobate perovskite.

11. The electrochemical sensor of claim 10, wherein the iron doped barium niobate perovskite comprises BaMgₙNb_{0.67-x}FeₓO_{3-δ} (BMNF) with a concentration of Fe from about x=0 to about x=0.50 and a concentration of Mg from about n=0.0 to about n=0.50.

12. The electrochemical sensor of claim 10, wherein the iron doped barium niobate perovskite comprises BaCaₙNb_{0.67-x}FeₓO_{3-δ} (BCNF) with a concentration of Fe from about x=0 to about x=0.50 and a concentration of Ca from about n=0.0 to about n=0.50.

13. The electrochemical sensor of claim 5, further comprises a gas separation membrane deposited onto the porous electrolyte stabilization layer.

14. The electrochemical sensor of claim 13, wherein the gas separation membrane comprising a material configured to transport hydrogen selectively to the first electrode and exclude cross interfering gases.

15. The electrochemical sensor of claim 1, further comprising mobile computing hardware to remotely transmit data.

16. A telemetric electrochemical sensor system, comprising:
a first electrode comprising LSCO, BMNF, or BCNF, a second electrode comprising a gold-palladium alloy, and a third electrode comprising platinum disposed on a first ceramic insulation layer;
a porous electrolyte stabilization layer disposed around and on top of the electrode;
a conductive ceramic substrate in contact with the first ceramic insulation layer on a first side;
a second ceramic insulation layer in contact with the conductive ceramic substrate on a second side; and
an integrated heater in contact with the second ceramic insulation layer; and
wherein the electrochemical sensor is configured to detect hydrogen, methane, or ammonia in a parts per million range.

17. A method of sensing gaseous analytes, comprising:
collecting a gaseous mixture sample containing a target analyte;
contacting the gaseous sample with a mixed potential electrochemical (MPE) sensor;
generating a signal based on a quantity of the target analyte in the gaseous mixture sample to quantify a concentration of the target analyte in the gaseous mixture sample; and
transmitting the concentration of the target analyte to a networked computing device.

18. The method of sensing gaseous analytes of claim 17, wherein the target analyte is hydrogen, methane, alkanes or ammonia, or a combination thereof.

19. The method of sensing gaseous analytes of claim 17, wherein a range of the concentration of the target analyte is in a parts per million (PPM) range.

20. The method of sensing gaseous analytes of claim 17, wherein contacting the mixed potential electrochemical (MPE) sensor comprises:
exposing the gaseous mixture sample to a hydrogen separation membrane disposed on top of a porous electrolyte comprising an iron (Fe) doped barium niobate perovskite;
establishing a first mixed potential (EMix 1) at a first electrode comprising LSCO, BMNF, or BCNF;
establishing a second mixed potential (EMix 2) at a second electrode comprising a gold-palladium alloy; and
determining a voltage difference (ΔE) as a sensing parameter to be measured.
